(19) United States
(12) Patent Application Publication
(10) Pub. No.: US 2006/0149139 A1
(43) Pub. Date: Jul. 6, 2006

(54) Apparatus and method for ascertaining and recording electrophysiological signals

(75) Inventors: Glorgio Bonmassar, Lexington, MA (US); John W Belliveau, Boston, MA (US)

Correspondence Address: DORSEY & WHITNEY LLP;INTELLECTUAL PROPERTY DEPARTMENT, 250 PARK AVENUE, NEW YORK, NY, 10177, US

(73) Assignees: GIORGIO BONMASSAR; JOHN W BELLLIVEAU

(21) Appl. No.: 10/535,958

(22) PCT Filed: Nov. 21, 2003
(86) PCT No.: PCT/US03/37761

§ 371 Date: Feb. 13, 2006

Related U.S. Application Data
(60) Provisional application No. 60/428,129, filed on Nov. 21, 2002.

(51) Int. Cl. A61B 5/00; A61B 5/02; A61B 5/103

(52) U.S. Cl. 600/300; 600/595; 600/504

(57) ABSTRACT

An arrangement and method for ascertaining and recording electrophysiological signals associated with a subject are provided. In particular, first (or motion) data associated with a movement of the subject from one or more motion sensors can be received. Such movement may include a head movement by the subject, swallowing by the subject, etc. The first data also can include noise associated with a blood flow motion within the subject, noise associated with a ballistocardiac motion within the subject, etc. Second data associated with intrinsic voltages measured may also be received from the subject. Then, output or result data can be calculated based on the first motion data and the second data. The output (or result data) is preferably associated with the electrophysiological signal. In an exemplary embodiment, a continuous, real time display of electrophysiological signals that is associated with the output data can be generated.

### CROSS-REFERENCE TO RELATED APPLICATION

The present application claims priority from U.S. Provisional Patent Application No. 60/428,129, which was filed on Nov. 21, 2002, and is entitled “Apparatus and Method for Ascertaining and Recording Electrophysiological Signals,” the disclosure of which is incorporated herein by reference in its entirety.

### FIELD OF THE INVENTION

The present invention relates generally to apparatus and method for ascertaining and recording electrophysiological signals. In particular, the invention is directed towards the apparatus and method in which various data associated with movements of the subject and data associated with intrinsic voltages measured from the subject are used to determine and/or record the electrophysiological signals.

### BACKGROUND OF THE INVENTION

Electrophysiological and functional magnetic resonance imaging (fMRI) provide complementary information about the timing and the location of processes occurring within a subject (e.g., a brain of the subject). Understanding the brain processes may include the acquisition of both electrophysiological and fMRI data. Specifically, neurons are cells specialized for the integration and propagation of electrical events, and it is through such electrical activity that neurons communicate with each other, muscles, and organs within the subject. Therefore, an understanding of basic electrophysiology can include understanding the functions and the dysfunctions of neurons, neural systems, and the brain. Moreover, if the particular brain process cannot be reproduced over multiple independent trials, it may be advantageous to simultaneously obtain electrophysiological and fMRI data to more readily understand the particular brain process. For example, sleeping stages, learning, and epileptic activity are brain processes which can be difficult to reproduce over multiple independent trials. Consequently, for a better understanding of the sleeping stages, learning, and epileptic activity within the subject, it may be advantageous to simultaneously obtain such electrophysiological and fMRI data.

Nevertheless, during electrophysiological recordings within a MRI environment, noise generally may be introduced into the electrophysiological signal. Specifically, the noise may be introduced by motion within the MRI environment during the recording of the electrophysiological signals. This noise may be associated with a ballistocardiogram motion (e.g., a cardiac pulsation) within the subject, a movement of the subject during the electrophysiological recording, etc. Moreover, the noise may obscure the electrophysiological signals at or below alpha frequencies (e.g., between about 8 Hz and 13 Hz). Specifically, in a magnetic field having a strength of about 1.5 T, the amplitude of the noise may be greater than or equal to about 150 μV, and the amplitude of the electrophysiological signals may be less than or equal to approximately 50 μV. Further, because these noises are present as a direct result of an electromagnetic induction in the magnetic field, the voltage differential between the amplitude of the noise and the amplitude of the electrophysiological signals may increase as the strength of the magnetic field increases.

One conventional method for removing the ballistocardiogram noise from the electrophysiological signal is to subtract an average ballistocardiogram waveform created based on the electrophysiological data (i.e., an average ballistocardiogram template) from the measured electrophysiological signal. Specifically, the average ballistocardiogram template may be created by averaging every electrophysiological channel, and using a linear regression to create the template. Nevertheless, over a predetermined period of time, a heart rate of the subject and/or a blood pressure of the subject may vary. Consequently, the amplitude and form of the ballistocardiogram noise signal also varies over the predetermined period of time. Such variations may be substantial, and can even occur during a one or more heart beats. As such, the average ballistocardiogram waveform may be inaccurate from one heart beat to the next, which can thus introduce systematic errors into the processed electrophysiological signals. Further, because the entire electrophysiological record may be relied upon to create this average ballistocardiogram waveform, the average ballistocardiogram waveform method may not be readily used to display continuous, real time electrophysiological signals. Moreover, the noise associated with the movement of the subject cannot be removed from the electrophysiological signals using the average ballistocardiogram waveform method.

### SUMMARY OF THE INVENTION

Therefore, a need has arisen to provide apparatus for recording electrophysiological signals associated with a subject and methods of ascertaining and recording such electrophysiological signals which overcome the above-described and other shortcomings of the related art. One of the advantages of the present invention is that electrophysiological signals may be determined and recorded in a MRI environment. Specifically, various noises associated with subject movements can be removed from data associated with intrinsic voltages measured from the subject in order to generate a display of electrophysiological signals. Another advantage of the present invention is that the noise associated with a ballistocardiogram motion within the subject and the noise associated with a blood flow motion within the subject also can be removed from the data associated with the intrinsic voltages to generate the display of electrophysiological signals.

According to an exemplary embodiment of the present invention, an arrangement and method for ascertaining and/or recording electrophysiological signals (e.g., electroencephalography (EEG) signals, electromyogram (EMG) signals, single and/or multi-cell signals, evoked potentials (EP) any other behavioral event signal, etc.) associated with a subject are provided. In particular, a processing system in the arrangement (e.g., a processing system associated with a computer system or a processing system associated with an EEG system) may be adapted to execute a filtering program. When the filtering program is executed, the processing system may be adapted (e.g., configured) to receive first data associated with a movement of the subject from one or more motion sensors (e.g., directly from the one or more motion sensors or indirectly from the one or more sensors via an analog to digital converter). Such movements may include head movements by the subject, swallowing by the subject, etc., and the first data may be amplified and then radio frequency (RF) filtered before processing system receives the first data. The motion data also can include noise associated with a blood flow motion within the subject, noise associated with a ballistocardiac motion within the subject, etc. The processing system also may be adapted to receive second data associated with intrinsic voltages measured from the subject (e.g., after the second data is amplified and RF filtered), and to calculate result data based on the first and second data, with the result data being associated with the electrophysiological signal. For example, the filtering program can include a filtering routine which receives the first data from the motion sensor, and generates an output which is subtracted from of the second data to generate the result data. Moreover, the processing system may further be adapted to generate a continuous, real time display of electrophysiological signals associated with the result data.

According to another exemplary embodiment of the present invention, a plurality of electrodes are positioned on at least one portion of the subject. An analog to digital (A/D) converter (e.g., a twenty-four (24) bit A/D converter) may be provided to be coupled to each of the electrodes. For example, the EEG system may include the A/D converter. The processing system can be coupled to the A/D converter. For example, the electrodes can be positioned on the scalp of the subject. The filter routine can be an adaptive filter routine, such as a Kalman-type adaptive filter routine. Moreover, the A/D converter is preferably adapted to measure intrinsic voltages associated with the subject, and to transmit the second data (which is associated with the intrinsic voltages) to the filtering program. In yet another exemplary embodiment of the present invention, the A/D converter can be positioned inside a MRI environment, and the processing system can be positioned outside the MRI environment. Alternatively, when the processing system is associated with the EEG system, the processing system can be positioned inside the MRI environment, and the computer system can be positioned outside the MRI environment. The motion sensor (e.g., a piezoelectric transducer) can provide signals and information to the filter routine (e.g., directly or via the A/D converter). For example, the motion sensor may be positioned adjacent to the subject, or on a portion of the subject (e.g., on a temporal artery of the subject). Further, at least one portion of the motion sensor may be filled with an acoustic dampening material, such as silicon, and can be adapted to measure the first data.

### BRIEF DESCRIPTION OF THE DRAWINGS

For a more complete understanding of the present invention, the needs satisfied thereby, and the objects, features, and advantages thereof, reference now is made to the following descriptions taken in connection with the accompanying drawings.

FIG. 1a is a schematic diagram of a first exemplary embodiment of an arrangement for recording electrophysiological signals associated with a subject according to the present invention.

FIG. 1b is a schematic diagram of a second exemplary embodiment of the arrangement according to the present invention.

FIG. 2 is a schematic diagram of a third exemplary embodiment of the arrangement according to the present invention.

FIG. 3a is a schematic diagram of a fourth exemplary embodiment of the arrangement according to the present invention.

FIG. 3b is a schematic diagram of a fifth exemplary embodiment of the arrangement according to the present invention.

FIG. 4a is a block diagram of an exemplary filtering program of the present invention that can be used in the arrangement of FIGS. 1a, 2, and 3a.

FIG. 4b is a block diagram of an exemplary filtering program of the present invention that can be used in the arrangement of FIGS. 1b and 3b.

FIG. 5 is a flowchart of a first exemplary embodiment of a method according to the present invention for recording electrophysiological signals associated with the subject.

FIG. 6 is a flowchart of a second exemplary embodiment of the method according to the present invention.

FIG. 7 is a flowchart of a third exemplary embodiment of the method according to the present invention.

FIG. 8 is a flowchart of a fourth exemplary embodiment of the method according to the present invention.

### DETAILED DESCRIPTION OF PREFERRED EMBODIMENTS

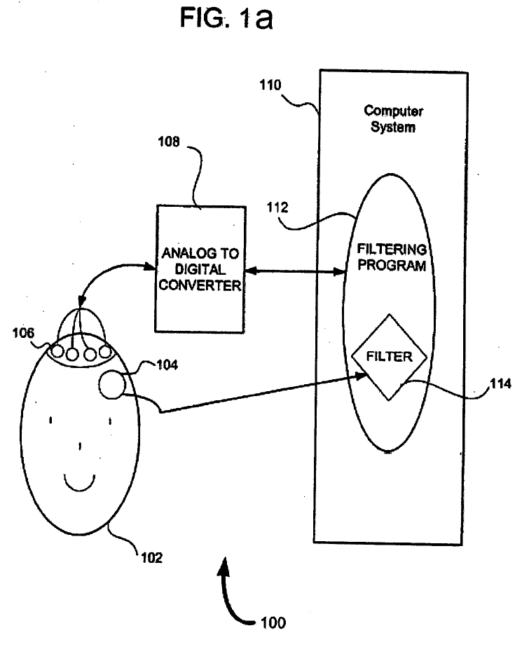

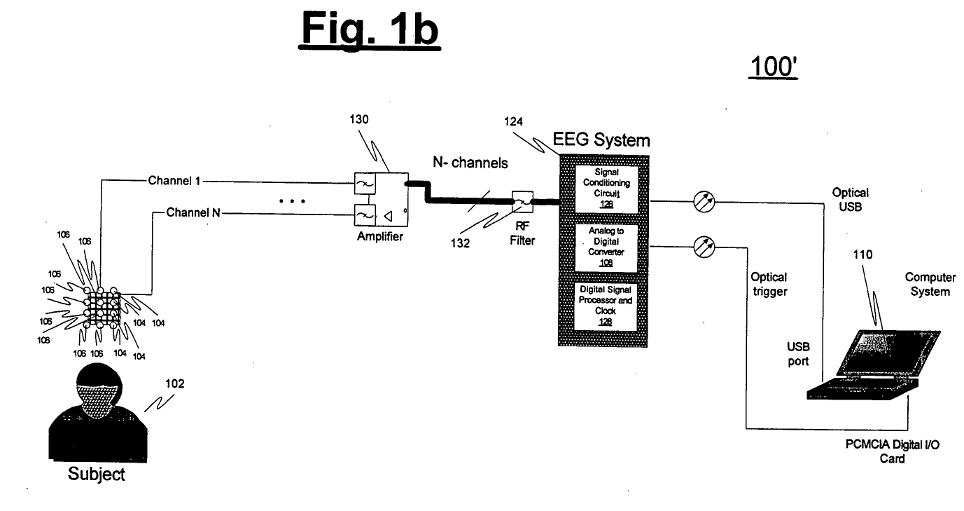

Preferred embodiments of the present invention and their advantages may be understood by referring to FIGS. 1a-8, like numerals being used for like corresponding parts in the various drawings.

Referring to FIG. 1a, a first exemplary embodiment of an arrangement 100 (100′) for recording electrophysiological signals (e.g., EEG signals, EMG signals, single and/or multi-cell signals, EP signals, any other behavioral event signal, etc.) associated with a subject 102 according to the present invention is provided. The arrangement 100 may include a plurality of electrodes 106 positioned on at least one portion of the subject 102 (e.g., a human being) For example, the electrodes 106 can be positioned along a scalp of the subject 102. A thirty-two channel MRI and electrophysiological compatible cap (not shown) can include the electrodes 106, and the cap may be positioned on a head of the subject 102. Alternatively, an eight channel electrophysiological set of plastic-conductive electrodes coated with a silver epoxy can be positioned along the scalp of the subject 102 using electrophysiological paste.

In an alternative embodiment of the present invention, e.g., for single cell recordings in an animal, electrodes 106 can be metal microelectrode held in a miniature micropositioner (not shown) on the head of the subject 102. For example, the micropositioner can be attached to a chronically implanted steel chamber or cylinder (not shown) that may be stereotaxically positioned and permanently cemented to the skull of the subject 102 in a prior surgery. These chambers may allow lateral repositioning of the microelectrode for multiple penetrations, while the micropositioner may allow the microelectrode to be lowered into the brain to a desired target along a particular track. Such microelectrodes can include (a) etched tungsten or platinum-iridium wires, insulated with either glass or lacquer except for ˜20 mm from the tip, (b) thin microwires that are typically 25-62 mm in diameter and lacquer-insulated except for the bluntly cut tip, etc. The type of microelectrode used may depend on the subject. For example, locus coeruleus neurons in awake rats and monkeys can be more easily recorded using the more flexible microwires. In particular, microwires can be advantageous for experiments entailing long-term recordings from neurons in deep structures in the subject 102, whereas etched, stiff microelectrodes are advantageous for studies where penetration of the dura mater may be needed or where numerous penetrations in a small area are desired.

The arrangement 100 also may include an analog to digital (A/D) converter 108 coupled directly or indirectly to the electrodes 106. For example, the A/D converter 108 can be a twenty-four bit A/D converter. Moreover, the A/D converter 108 may be adapted to measure intrinsic voltages associated with the subject 102.

Referring to FIG. 1a, in another embodiment of the present invention, the arrangement 100′ may include an EEG system 124, and the EEG system 124 may include the A/D converter 108. In this embodiment, the EEG system 124 also may include a programable, signal conditioning circuit 126, and a digital signal processor (DSP) 128, each of which may be coupled to the A/D converter 108. Further, the electrodes 106 may be coupled to an amplifier 130, the amplifier 130 may be coupled to an radio frequency (RF) filter 132, and the RF filter 132 may be coupled to the EEG system 124. For example, the RF filter 132 may be coupled to the programable, signal conditioning circuit 126, and the programable, signal conditioning circuit 126 may be coupled to the A/D converter 108. Moreover, the DSP processor 128 may determine the number of channels which are dedicated to obtaining the electrophysiological signals from the subject 102 by adjusting the sample rate. For example, the DSP processor 128 may increase the number of channels dedicated to obtaining the electrophysiological signals by decreasing the sample rate, or vice versa (e.g., if a desired number of channels dedicated to obtaining the electrophysiological signals is 32 channels, the sample rate may be 1000 sampled per second).

Referring again to FIG. 1a, the arrangement 100 may further include a processing system 110 coupled to the A/D converter 108. The processing system 110 may be adapted to execute a filtering program 112, that may be resident on its storage device (or on an external storage device), which may include a filter routine 114. For example, the filter routine 114 can be an adaptive filter routine, such as a Kalman-type adaptive filter routine. Moreover, the processing system 110 may be adapted (e.g., by executing the filtering program 114) to receive data associated with the intrinsic voltages from the A/D converter 108.

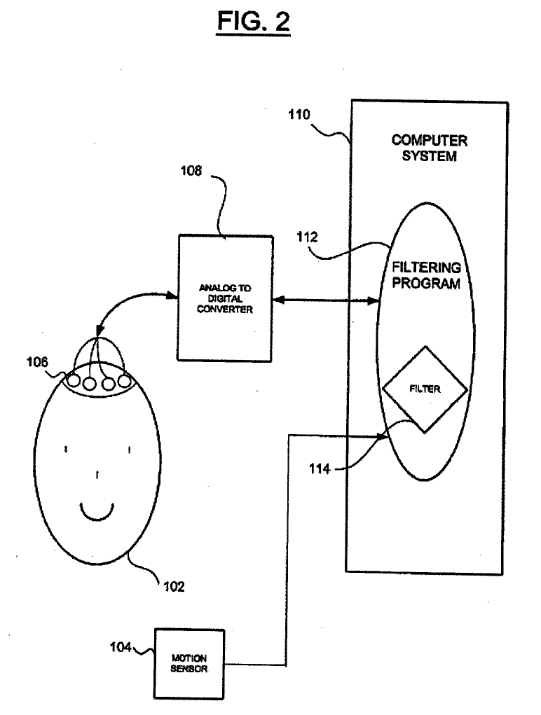

Referring again to FIG. 1a, the arrangement 100 also may include one or more motion sensors 104 coupled directly or indirectly to the filter routine 114 of the filtering program 112. For example, the one or more motion sensors 104 may be a piezoelectric transducer. At least one portion of the one or more motion sensors 104 may be filled with an acoustic dampener (not shown), such as silicon, which may allow the one or more motion sensors 104 to be less sensitive to acoustic noise. In an exemplary embodiment of the present invention, the one or more motion sensors 104 may be positioned on at least one portion of the subject 102, such as on a temporal artery of the subject 102. Referring back to FIG. 2, the one or more motion sensors 104 can be positioned adjacent to the subject 102.

Referring to FIG. 1b, in another embodiment of the present invention, the electrophysiological compatible cap (not shown) can include the electrodes 106 and the one or more motion sensors 104. For example the electrophysiological compatible cap may include about twenty eight (28) electrodes and about four (4) motion sensors. The electrodes 106 and the one or motion sensors 104 each may be coupled to the amplifier 130, the RF filter 132, the signal conditioning circuit 126, the A/D converter 108, and the filtering program 112. Moreover, in this embodiment, the filtering program 112 may be executed by the processing system 110 and/or the DSP 128.

In the exemplary embodiments of the present invention, the one or more motion sensors 104 may be adapted to measure motion data associated with a movement of the subject 102. Such movements may include head movements by the subject, swallowing by the subject, etc. Nevertheless, it should be understood by those having ordinary skill in the art that the movements can include any movements of the subject 102 which may introduce noise into the intrinsic voltages being received and/or measured. The motion data also can include noise associated with a blood flow motion within the subject, noise associated with a ballistocardiac motion (e.g., a cardiac pulsation) within the subject, etc. Moreover, when the filter routine 114 is executed by the processing system 110, the measured motion data originating from the one or more motion sensors 104 can be received by such processing system 110.

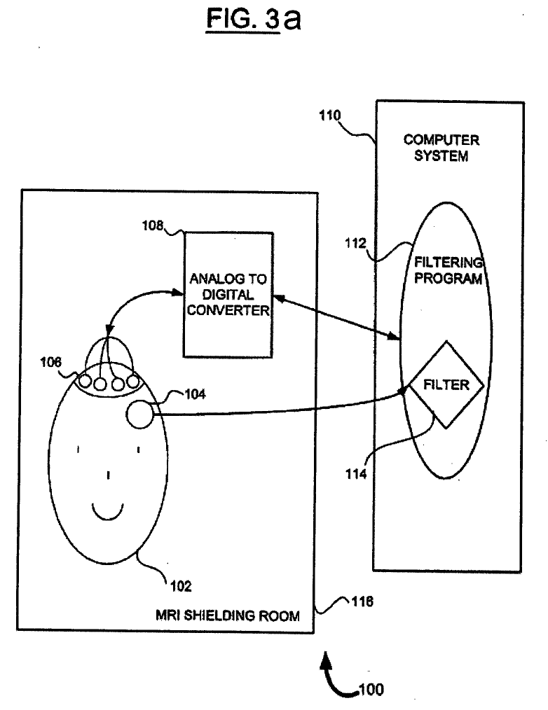

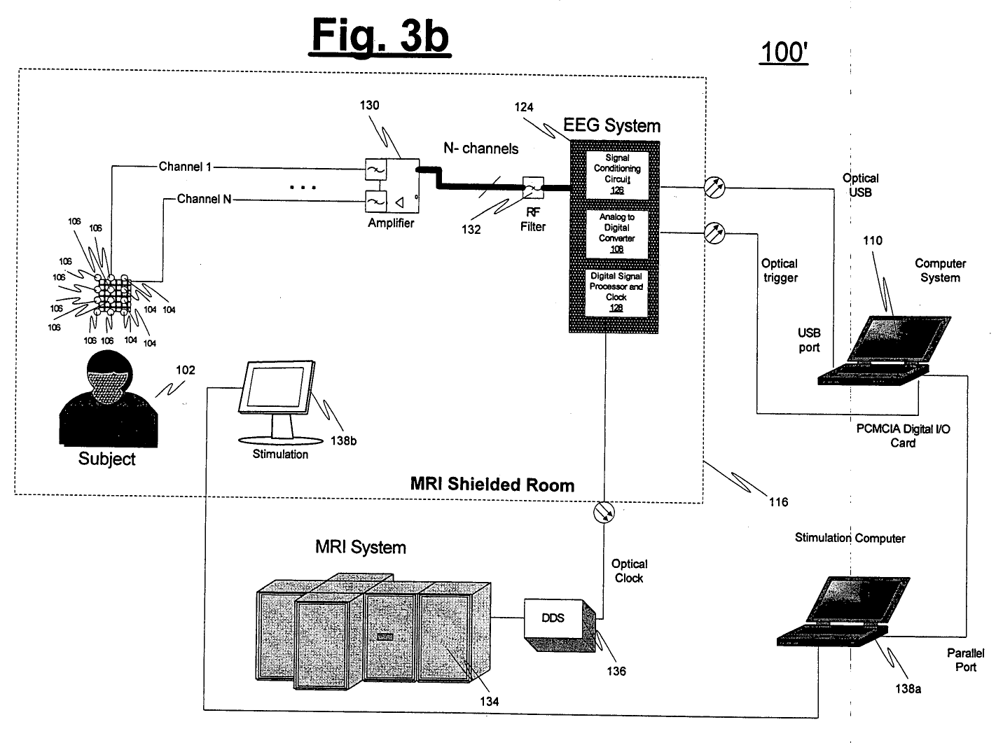

FIG. 3a shows another exemplary embodiment of the arrangement 100 according to the present invention, which may be adapted to simultaneously record the electrophysiological signals and fMRI signals. Specifically, the A/D converter 108, the subject 102, the one or more motion sensors 104, and the electrodes 106 can be positioned inside an MRI shielding room 116, and the processing system 110 can be positioned outside the MRI shielding room 116. Further, the A/D converter 108 can be coupled to the processing system 110 using an optical cable or by any other wired or wireless connection.

FIG. 3b shows another exemplary embodiment of the arrangement 100′ according to the present invention, which also may be adapted to simultaneously record the electrophysiological signals and fMRI signals. Specifically, the EEG system 124, the subject 102, the one or more motion sensors 104, the electrodes 106, the amplifier 130, and the RF filter can be positioned inside the MRI shielding room 116, and the processing system 110 can be positioned outside the MRI shielding room 116. In this embodiment of the present invention, the arrangement 100′ can also include a MRI system 134 coupled to a direct digital synthesizer (DDS) 136, and the DDS may be coupled to the DSP 128. Moreover, the MRI system 134 may include a first clock, and the DSP 128 may include a second clock. Specifically, the DDS 136 may synchronize the first clock of the MRI system 134 with the second clock of the DSP system 128. For example the DDS 136 may convert a sine wave output from the MRI system 134 (e.g., 10 MHz sine wave output) into a square wave signal (e.g., a 48 MHz square wave) to filter out a scanning noise associated with the arrangement 100′. Further, the amplifier 130 and the RF filter 132 each may be enclosed in a plastic box having a conductive aluminum coating, which may prevent eddy currents from affecting the amplifier 130 and the RF filter 132. In another embodiment, the connectors between each of the elements within the MRI shielding room 116 may include a capacitor array to complete the RF shielding, and some or all of the elements within the MRI shielding room 116 may be battery operated to reduce noise. Moreover, some or all of the cables used to connect the elements inside the MRI shielding room 116 may be plastic fiber optic cables. The arrangement 100′ also may include one or more stimulation computers 138 (e.g., a pair of stimulation computers 138a and 138b) for delivering sound, images, and/or tactile information to the subject 102. The one or more stimulation computers 138 may be coupled to the computer system 110 (e.g., via a parallel port), and also may be adapted to transmit a signal (e.g., an eight (8) bit signal) to the computer system 110 each time a stimulus is transmitted to the subject 102. For example, a first image transmitted to the subject 102 may be coded as a binary 0001, a second image may be coded as a binary 0002, a first sound may be coded as a binary 0003, a second sound may be coded as a binary 0004, etc. This may be particularly useful when evoked potentials are being observed.

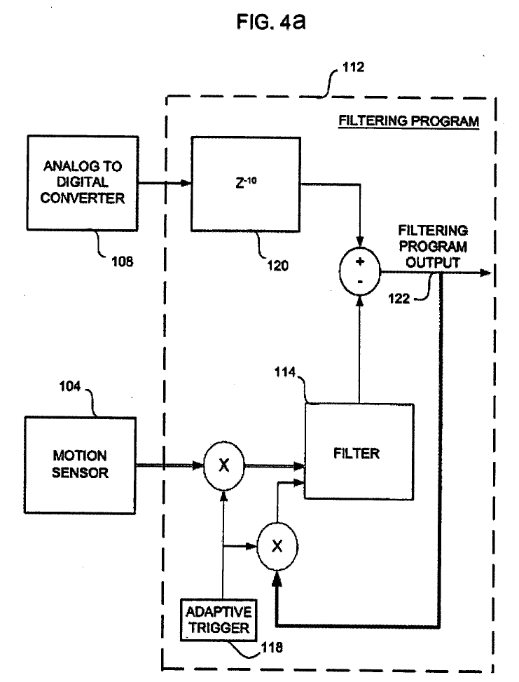

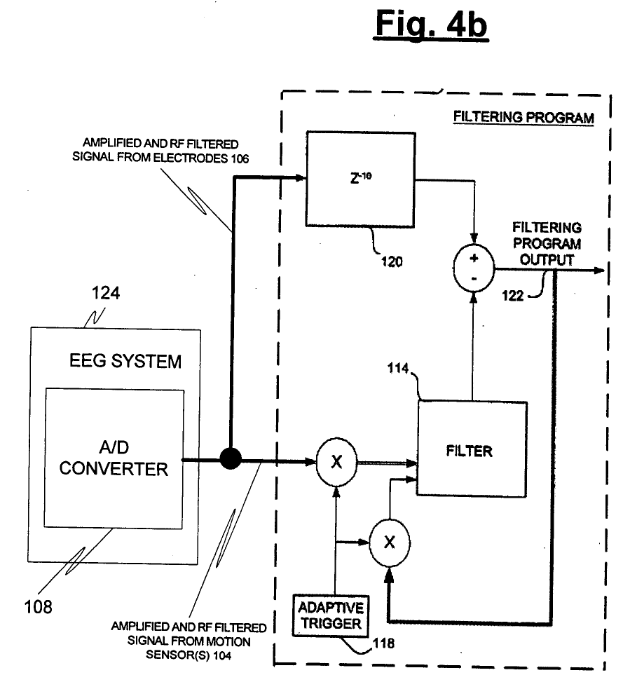

FIG. 4a shows a block diagram of an exemplary embodiment of the filtering program 112 which can be used in the exemplary embodiments of the arrangements 100 depicted in FIGS. 1a, 2, and/or 3a, and FIG. 4b shows a block diagram of an exemplary embodiment of the filtering program 112 which can be used in the exemplary embodiments of the arrangements 100′ depicted in FIGS. 1b and/or 3b. It will be understood by those of ordinary skill in the art that the filtering program shown in FIGS. 4a and 4b may be substantially the same, except that the signals received by the filtering program 112 in FIG. 4b may be amplified and RF filtered signals. The filtering program 112 includes the filter routine 114, and as an input to the filter routine 114 (when executed by the processing system), the motion data associated with the output of the one or more motion sensors 104 is provided. The data provided to the filtering program 112 from the one or more motion sensors 104 may be forwarded from an adaptive trigger 118 which modulates the transmission of the output from the one or more motion sensors 104 into the motion data. The filtering program 112 further includes a time delay block 120 receiving information from the A/D converter 108. Specifically, the time delay block 120 may be used to compensate for an intrinsic delay within the one or more motion sensors 104 caused by mechanical inertia. For example, the time delay block 120 may delay the transmission of the data associated with the intrinsic voltages from the A/D converter 108 by about 100 ms. In operation, the filter routine 114 can process the motion data received from the one or more motion sensors 104, and the output of the filter routine 114 and the data associated with the measured intrinsic voltages can be transmitted to a summation block. Further, the filtering program 112 may be adapted to subtract the output of the filter routine 114 from the measured intrinsic voltages in order to obtain output data 122 of the filtering program 112. The output data 122 may be provided as input of the filter routine 114 to complete a feedback loop of the filtering program 112 shown in FIG. 4a and/or FIG. 4b. Moreover, the processing system 110 may be adapted to generate a continuous, real time display of electrophysiological signals associated with the output data 122, including a display of those electrophysiological signals at or below the alpha frequencies (e.g., between 8 Hz and 13 Hz.)

The intrinsic voltages measured by the A/D converter 108 likely include both the electrophysiological signals and the noise signals associated with motion of the subject 102. Specifically, the intrinsic voltages include the electrophysiological signals and the noise signals associated with the movements of the subject 102, the blood flow motion within the subject 102, and the ballistocardiac motion within the subject 102. Nevertheless, the one or more motion sensors 104 can be positioned so that the one or more motion sensors 104 substantially measures only the signals associated with noise (e.g., the same noise signals included in the measured intrinsic voltages). Consequently, according to an exemplary embodiment of the present invention, when the filtering program 112 subtracts the data associated with the measured intrinsic voltages from the data provided at the output of the filter routine 114, particular data associated with the electrophysiological signals remain in the output data 122 (either with other data or without any other data). Subsequently, the processing system 110 can generate a continuous, real time display of electrophysiological signals associated with the output data 122.

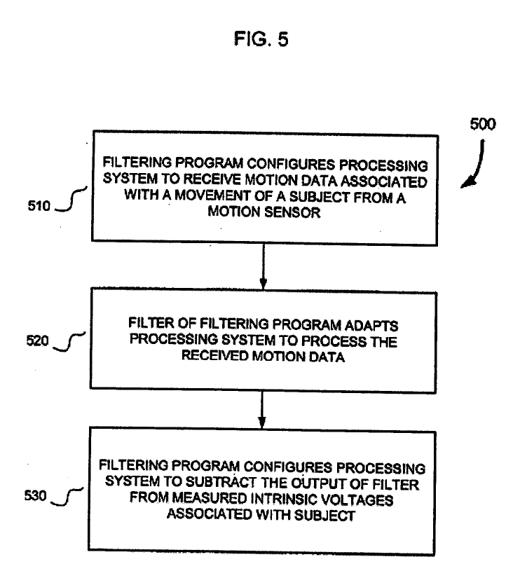

FIG. 5 shows a first exemplary embodiment of a method 500 according to the present invention for ascertaining and recording the electrophysiological signals associated with the subject 102. In step 510, the processing system 110 receives the motion data associated with the movement of the subject 102 from the one or more motion sensors 104. In step 520, the processing system 110 receives the data associated with intrinsic voltages measured from the subject 102. Then, in step 530, the processing system 110 executes the filtering program 112 and calculates the output (or result) data (associated with the electrophysiological signals) based on the received motion data and the data associated with the intrinsic voltages.

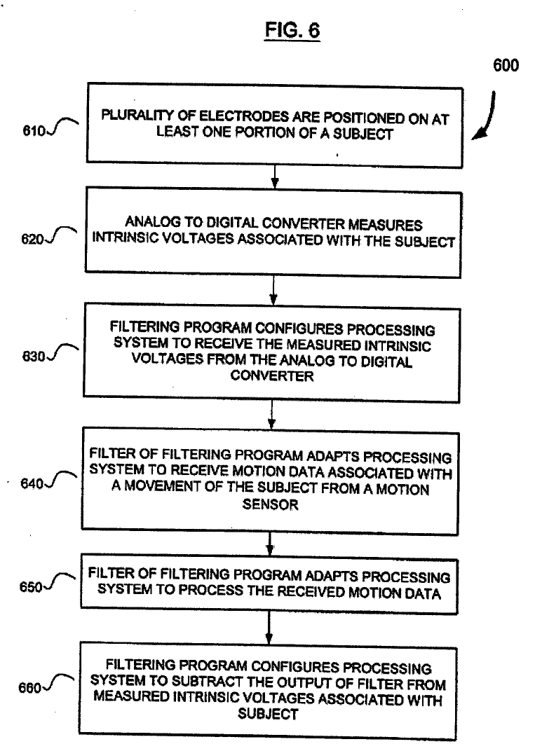

FIG. 6 shows a second exemplary embodiment of a method 600 according to the present invention for ascertaining and recording the electrophysiological signals associated with the subject. In this embodiment, the electrodes 106 are positioned on at least one portion of the subject 102 (step 610). In step 620, the A/D converter 108 measures the intrinsic voltages associated with the subject 102. The processing system 110 receives the data associated with the measured intrinsic voltages from the A/D converter 108 in step 630. In step 640, the processing system 110 receives the motion data associated with the movement of the subject 102 from the one or more motion sensors 104. Then, in step 650, the processing system 110 executes the filtering program 112 to calculate the output or result data (as defined above) based on the received motion data and the data associated with the intrinsic voltages.

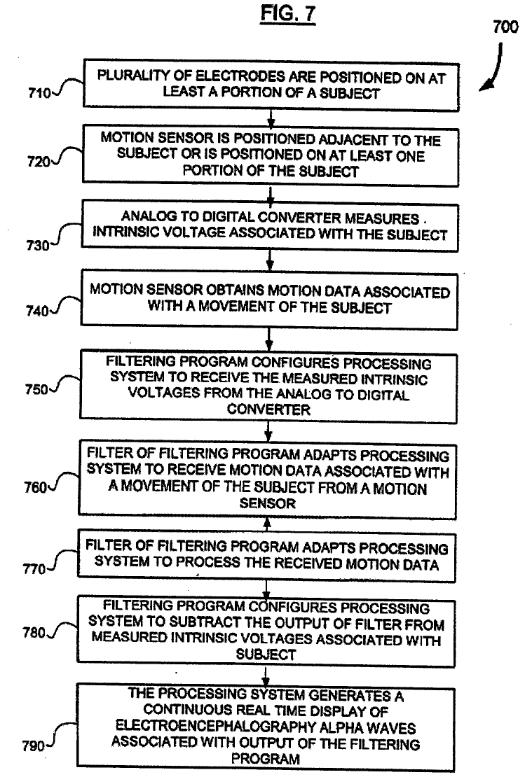

Referring to FIG. 7, a third exemplary embodiment of a method 700 according to the present invention for ascertaining and recording the electrophysiological signals is provided. In step 710, the electrodes 106 are positioned on at least one portion of the subject 102. In step 720, the one or more motion sensors 104 may be positioned adjacent to the subject 102 or on at least one portion of the subject 102 (e.g., on either the same or different portion as that on which the sensor 104 is situated). In step 730, the A/D converter 108 measures the intrinsic voltages associated with the subject 102, and in step 740, the one or more motion sensors 104 measures the motion data associated with a movement of the subject 102. The filtering program 112, when executed, configures the processing system 110 to receive the data associated with the measured intrinsic voltages from the A/D converter 108 in step 750. The filter routine 114 of the filtering program 112 then enables the processing system 110 to receive the motion data from the one or more motion sensors 104 (step 760). In step 770, the filter routine 114 configures the processing system 110 to process the received motion data, and in step 780, such configured processing system 110 subtracts the output generated by the filter routine 114 from the data associated with the measured intrinsic voltages. Moreover, in step 790, the processing system 110 generates a continuous, real time display of the electrophysiological signals associated with the output or result data 122.

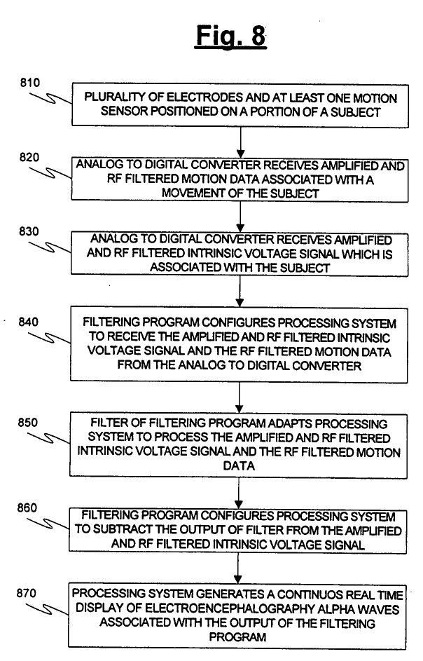

Referring to FIG. 8, a fourth exemplary embodiment of a method 800 according to the present invention for ascertaining and recording the electrophysiological signals is provided. In step 810, electrodes 106 and the one or more motion sensors 104 are positioned on a portion of the subject 102. In step 820, the A/D converter receives amplified and RF filtered motion data associated with a movement of the subject 102 from the one or more motion sensors 104. In step 830, the A/D converter receives amplified and RF filtered intrinsic voltage signal which is associated with the subject 102. In step 840, the filtering program 112 configures the processing system 110 and/or the DSP 128 to receive the amplified and RF filtered motion data and the amplified and RF filtered intrinsic voltage signal from the A/D converter 108. Moreover, in step 850, the filter 114 adapts the processing system 110 and/or the DSP 128 to process the amplified and RF filtered motion data and the amplified and RF filtered intrinsic voltage signal. In step 860, the filter 114 adapts the processing system 110 and/or the DSP 128 to subtract the output of the filter 114 from the amplified and RF filtered intrinsic voltage signal. Further, in step 870, the processing system 110 and/or the DSP 128 generates a continuous real time display of electroencephalography alpha waves associated with the output of the filter program 112.

While the invention has been described in connection with preferred embodiments, it will be understood by those of ordinary skill in the art that other variations and modifications of the preferred embodiments described above may be made without departing from the scope of the invention. Other embodiments will be apparent to those of ordinary skill in the art from a consideration of the specification or practice of the invention disclosed herein. It is intended that the specification and the described examples are considered as exemplary only, with the true scope and spirit of the invention indicated by the following claims.

What is claimed is:

1. An arrangement for ascertaining and recording an electrophysiological signal associated with a subject, comprising:
a processing system which, when executing a filtering program, is adapted to:
receive first data associated with a movement of the subject from at least one motion sensor,
receive second data associated with at least one intrinsic voltage measured from the subject, and
calculate result data based on the first data and the second data, wherein the result data is associated with the electrophysiological signal.

2. The arrangement of claim 1, wherein the first data is motion data for at least one of a head movement and a swallowing motion by the subject.

3. The arrangement of claim 1, wherein the filtering program comprises a filter routine.

4. The arrangement of claim 3, wherein the filter routine is adapted to receive and process the motion data.

5. The arrangement of claim 4, wherein the first data is an output of the filter routine, and wherein the result data is obtained by subtracting the first data from the second data.

6. The arrangement of claim 5, wherein the filter routine is an adaptive filter routine.

7. The arrangement of claim 6, wherein the adaptive filter routine is a Kalman-type adaptive filter routine.

8. The arrangement of claim 1, further comprising:
a plurality of electrodes positioned on at least one portion of the subject; and
an analog to digital (A/D) converter coupled to each of the electrodes, wherein the A/D converter is adapted to measure the at least one intrinsic voltage and transmit the first data to the processing system.

9. The arrangement of claim 8, wherein the A/D converter is a 24 bit A/D converter.

10. The arrangement of claim 8, wherein the A/D converter is positioned within a magnetic resonance imaging environment, and wherein the processing system is positioned outside the magnetic resonance imaging environment.

11. The arrangement of claim 8, wherein the at least one motion sensor is a piezoelectric transducer positioned on a temporal artery of the subject, and wherein each of the electrodes is positioned on a scalp portion of the subject.

12. The arrangement of claim 1, wherein the at least one motion sensor includes at least one portion which is filled with an acoustic dampener that has silicon.

13. The arrangement of claim 1, wherein the first data is further associated with at least one of a ballistocardiac motion within the subject and a blood flow motion within the subject.

14. The arrangement of claim 13, wherein the ballistocardiac motion includes a cardiac pulsation.

15. The arrangement of claim 1, wherein the processing system is further adapted to generate a continuous, real time display of the electrophysiological signals associated with the result data.

16. The arrangement of claim 1, wherein the at least one motion sensor has an output which is modulated by an adaptive trigger.

17. The arrangement of claim 1, further comprising:
a plurality of electrodes positioned on at least one portion of the subject;
an amplifier coupled to each of the at least one motion sensor and the plurality of electrodes;
an RF filter coupled to the amplifier; and
an analog to digital (A/D) converter coupled to the RF filter, wherein the A/D converter is adapted to measure the at least one intrinsic voltage and transmit the first data to the processing system.

18. The arrangement of claim 17, wherein the RF filter is coupled to the A/D converter via a programable, signal conditioning circuit.

19. The arrangement of claim 18, wherein the at least one motion sensor is coupled to the RF filter, such that the at least one motion sensor is coupled to the processing system via the amplifier, the RF filter, the signal conditioning circuit, and the A/D converter.

20. The arrangement of claim 19, further comprising an EEG system, wherein the EEG system comprises the signal conditioning circuit, the A/D converter, and a digital signal processor, and wherein the digital signal processor comprises a first clock.

21. The arrangement of claim 20, further comprising a MRI system comprising a second clock, wherein an output of the MRI system is connected to the digital signal processor via a direct digital synthesizer, and wherein the direct digital synthesizer is adapted to synchronize the second clock with the first clock.

22. The arrangement of claim 21, further comprising a stimulation system for stimulating the subject.

23. A method of ascertaining and recording electrophysiological signals associated with a subject, comprising the steps of:
receiving first data associated with a movement of the subject from at least one motion sensor;
receiving second data associated with at least one intrinsic voltage measured from the subject; and
calculating result data based on the first motion data and the second data using a filtering routine, wherein the result data is associated with the electrophysiological signal.

24. The method of claim 23, wherein the first data is motion data for at least one of a head movement and a swallowing motion by the subject.

25. The method claim 23, wherein a processing system is adapted to receive the motion data associated with the movement of the subject, and wherein the filtering routine adapts a processing system to receive the second data.

26. The method of claim 25, wherein the first data is an output of the filter routine, and wherein the calculating step comprises subtracting the first data from the second data.

27. The method of claim 23, wherein at least one portion of the at least one motion sensor includes an acoustic dampener comprising silicon.

28. The method of claim 23, wherein the filter routine is an adaptive filter routine.

29. The method of claim 28, wherein the adaptive filter routine is a Kalman-type adaptive filter routine.

30. The method of claim 23, further comprising the steps of:
positioning a plurality of electrodes on at least one portion of the subject;
measuring the at least one intrinsic voltage using an analog to digital (A/D) converter; and
transmitting the first data to the processing system.

31. The method of claim 30, wherein the A/D converter is a 24 bit A/D converter.

32. The method of claim 30, wherein the A/D converter is positioned within a magnetic resonance imaging environment, and wherein the processing system is positioned outside the magnetic resonance imaging environment.

33. The method of claim 30, wherein the at least one motion sensor is a piezoelectric transducer positioned on a temporal artery of the subject, and wherein each of the electrodes are positioned on the scalp of the subject.

34. The method of claim 23, wherein the first data is further associated with at least one of a ballistocardiac motion within the subject and a blood flow motion within the subject.

35. The method of claim 34, wherein the ballistocardiac motion includes a cardiac pulsation.

36. The method of claim 23, further comprising the step of generating a continuous, real time display of the electrophysiological signals associated with the result data.

37. The method of claim 23, further comprising the step of modulating an output of the at least one motion sensor.
